(19) United States
(12) Patent Application Publication
(10) Pub. No.: US 2018/0267066 A1
(43) Pub. Date: Sep. 20, 2018

(54) LIPODOMIC BIOMARKERS OF METABOLIC DISEASES IN DAIRY COWS

(71) Applicant: Brigham Young University, Provo, UT (US)

(72) Inventors: Beverly L. ROEDER, Sandy, UT (US); Steven William GRAVES, Highland, UT (US)

(73) Assignee: Brigham Young University, Provo, UT (US)

(21) Appl. No.: 15/763,952

(22) PCT Filed: Sep. 28, 2016
(86) PCT No.: PCT/US2016/054153

§ 371 Date: Mar. 28, 2018

Related U.S. Application Data
(60) Provisional application No. 62/233,905, filed on Sep. 28, 2015.

(51) Int. Cl. G01N 33/92; G01N 33/487; G01N 27/62

(52) U.S. Cl. CPC G01N 33/92; G01N 33/487; G01N 27/62; G01N 2800/60; G01N 2800/50; G01N 2800/56; G01N 2800/04

(57) ABSTRACT

Lipid biomarkers for early detection of production-related metabolic disease in cattle are disclosed. The biomarkers are obtained from colostrum or milk. Methods for using and analyzing such lipid biomarkers, and methods for managing and treating dairy cattle are disclosed.

### RELATED APPLICATIONS

This application claims the benefit under 35 U.S.C. § 119(e) of Provisional U.S. Patent Application No. 62/233,905, filed Sep. 28, 2015, the contents of which are hereby incorporated by reference, in their entirety.

### BACKGROUND

### 1. Technical Field

The application relates to biomarkers of metabolic disease in cattle. More particularly, the application relates to lipid biomarkers in colostrum and milk that allow early detection of production-related metabolic disease in asymptomatic dairy cattle, methods for using and analyzing such lipid biomarkers, and methods for managing and treating dairy cattle.

### 2. Background Information

Production-related metabolic diseases (PRMDs) are debilitating disorders occurring during early lactation in dairy cows. PRMDs are multifactorial and the causes are complex. Different phenotypes of PRMDs have been documented based on the clinical signs manifested. However, although a variety of health problems have been identified, they are currently thought to be interrelated physiologically, and not distinct, independent entities. For example, acute milk fever results from hypocalcemia, characterized by reduced ionized blood calcium levels, and is treated as a medical emergency. Displaced abomasum, in contrast, has been shown to be preceded by ketosis, and is often accompanied by subclinical hypocalcemia. Ketosis is a metabolic process that occurs when the animal does not have enough glucose to maintain normal cellular energy production. Consequently, stored fats are broken down and mobilized to meet energy needs, resulting in elevated fatty acids and a build-up of organic acids, termed ketone bodies, in the animal's circulation. Fatty liver syndrome in dairy cows has been shown to occur when the synthesis of triacylglycerols is higher than their export and appears to be associated with hormone dysregulation. Disease states associated with ketosis may also be associated with varying degrees of hepatic lipidosis.

Any of the aforementioned PRMDs may occur with concurrent mastitis, metritis, retained placenta, or other health problems. Unfortunately, the incidence of many PRMDs is not effectively altered by management with transition diets, dietary cation-anion manipulation, or avoidance of over conditioning.

For these and other reasons, intervention for many PRMDs is currently available only after onset of clinical signs, resulting in additional economic cost associated with medication and labor. Since PRMDs are not limited to a single phenotype and represent an array of disorders having interrelated causes, including dietary and environmental management practices, there is a need in the art to identify and predict downstream problems in milk production associated with these metabolic disorders, thereby allowing appropriate interventions.

PRMDs have been correlated with elevated serum concentrations of free fatty acids (FFAs), non-esterified fatty acids (NEFAs), triglycerides (TG), β-hydroxybutyric acid (Oetzel 2004), and hepatic TG:glycogen ratios (Petit et al, 2007; Yamamoto et al, 2001). Milk fatty acids, such as C18:1 cis-9, have also been studied as possible biomarkers to diagnose elevated concentrations of plasma NEFA (Jorjong et al, 2014) and hyperketonemia (Jorjong et al, 2015) in lactating dairy cows. Prior evaluations of postpartum cattle for lipid biomarkers of PRMDs have looked at milk 2, 3, 4, and 8 weeks post-partum for markers of hyperketonemia (Jorjong et al, 2014). However, these biomarkers typically present only after symptoms appear, and require invasive, time-consuming and expensive sampling.

Earlier detection of biomarkers for PRMD will allow breeders to identify cattle at risk for developing disease, to potentially separate or treat these animals, saving significant time and money. It would be particularly useful to identify biomarkers in milk or colostrum that can be evaluated before symptoms present.

### BRIEF SUMMARY

In one aspect, methods are provided for managing dairy cattle, including sampling colostrum or milk from a cow 0-7 days after parturition; analyzing the sample for levels of one or more lipid markers; identifying a cow at increased risk of production-related metabolic disease (PRMD) based on the one or more lipid markers; and separating the cow.

In one aspect, methods are provided for determining risk for production-related metabolic disease (PRMD) in cattle, including obtaining a colostrum or milk sample from a post-partum cow 0-7 days after parturition; analyzing the sample for levels of one or more lipid markers; and determining if the cow is at increased risk of PRMD.

In embodiments, the colostrum is sampled 0 to 24 hours after parturition. In embodiments, the milk is sampled 4 to 7 days after parturition. In some embodiments, the animal determined to be at risk of PRMD is treated. In one aspect, a panel of lipid biomarkers in colostrum and/or milk is disclosed.

In embodiments, the lipid markers are analyzed using mass spectrometry. In some embodiments, the lipid markers are analyzed using electrospray injection time-of-flight mass-spectrometry (ESI-TOF MS).

In some embodiments, the lipid markers in colostrum are selected from the group of lipid markers having a mass to charge ratio of about 344.23, 570.46, 586.54, 652.55, 682.56, 855.75, 919.83, and combinations thereof, as determined by electrospray injection time-of-flight mass-spectrometry in ammonium acetate. In embodiments, the lipid markers have an elemental composition selected from the group consisting of C₄₀H₅₆O+NH₄⁺, C₃₅H₆₈O₅+NH₄⁺, C₅₅H₉₈O₆+H⁺, C₄₁H₇₆O₆+NH₄⁺, C₂₁H₂₆O₃+NH₄⁺, C₄₀H₇₄O₅+NH₄⁺, C₅₈H₁₁₀O₆+OH, and combinations thereof. In embodiments, the lipid markers are selected from the group consisting of DG (16:0/16:0), TG (16:0/18:1/18:3), DG (18:2/19:0), an oxidized TG (18:0/18:0/19:1)+OH, an oxidized TG (18:0/18:1/19:0)+OH, and combinations thereof.

In some embodiments, the lipid marker samples in milk are selected from the group of lipid markers having a mass to charge ratio of about 642.56, 652.55, 740.67, 906.84, 919.83, and combinations thereof, as determined by ESI-TOF MS. In embodiments, the lipid markers have an elemental composition selected from the group consisting of C₃₉H₇₆O₅+NH₄⁺, C₅₇H₁₀₈O₆+NH₄⁺, C₄₅H₈₆O₆+NH₄⁺, C₅₈H₁₁₀O₆+OH, C₄₀H₇₄O₅+NH₄⁺, and combinations thereof. In some embodiments, the lipid markers are selected from the group consisting of DG (18:0/18:0), TG (18:0/18:0/18:1), TG(12:0/14:0/16:0), oxidized TG (18:0/18:0/19:1)+OH, oxidized TG (18:0/18:1/19:0)+OH, DG(18:2/19:0), and combinations thereof.

### BRIEF DESCRIPTION OF THE DRAWINGS

FIG. 1 presents six mass spectra from six colostrum samples.

FIG. 2 is a plot of linear discriminative analysis results for lipid biomarkers.

### DETAILED DESCRIPTION

Disclosed herein are various exemplary embodiments of the invention. The following embodiments are not meant to limit the invention or narrow the scope thereof, as it will be readily apparent to one of ordinary skill in the art that suitable modifications and adaptations may be made without departing from the scope of the invention. All patents and publications cited herein are incorporated by reference for the specific teachings thereof.

Terms such as “include,” “including,” “contain,” “containing,” “has,” or “having” and the like mean “comprising.” Headings herein are provided for the convenience of the reader and for navigation throughout the document, and are not intended to limit the scope of the subject matter described thereafter.

Production-related metabolic diseases (PRMDs) continue to be a costly problem for the dairy industry. Different phenotypes of PRMDs have been documented, including hypocalcemia (commonly known as milk fever) (MF), left displaced abomasum (LDA), ketosis, obturator nerve paresis (OP), fatty liver syndrome, and retained placenta among the most common disorders. These health problems may present with decreased milk production, altered milk composition, reduced reproductive capacity, shortened life expectancy, and/or lower cull value, resulting in both animal and economic loss.

The treatment of PRMDs, once the disease is established, can involve intensive dietary supplementation, drug therapy, or even surgery, but there may still be loss of the animal depending on how late the intervention or how severe the disease at the time of diagnosis.

Prevention of PRMDs is likely possible and would be a more effective and cheaper strategy than medical treatment of active disease. This, however, requires a reliable and easy way of predicting cows at risk for PRMDs in advance of clinical signs. Identifying animals that will have complications early enough to prevent onset of PRMDs has not previously been possible.

Most PRMDs occur shortly after calving during the first few days to weeks of lactation, and are believed to be due to difficulty adapting to the high demands of lactation, resulting in physiological imbalances in susceptible cows.

Lipid metabolism has been reported to be abnormal in certain metabolic diseases in dairy cows. For example, the liver triglyceride to glycogen ratio has been used to predict susceptibility of cows to ketosis. Increases in plasma non-esterified fatty acids (NEFA) and β-hydroxybutyrate (BHBA) were both significantly associated with development of peripartum diseases. Milk fatty acids, such as C18:1 cis-9, have been proposed as possible biomarkers able to diagnose elevated concentrations of plasma NEFA early in dairy cows. However, there has not been a comprehensive study of lipid biomarkers with the intent of their predicting PRMDs.

Previous attempts to identify biomarkers of PRMDs have focused on levels of lipids in plasma or serum. For example, animals with an elevated serum NEFA concentration (more than 0.3 mEq/L) between 14 to 2 days prior to calving, or animals with an elevated serum β-hydroxybutyrate concentration (more than 10 mg/dL) and NEFA concentration (more than 0.6 mEq/L) 3 to 14 days postpartum, have been shown to be at somewhat greater risk of transition PRMDs in dairy cows (Ospina et al.). However, the sensitivity and specificity of these existing markers were inadequate to be useful. Moreover, collection of blood samples by venipuncture is labor intensive, expensive and impractical as a screening tool. Therefore, it is desirable to discover novel biomarkers that predict PRMDs with adequate sensitivity and specificity.

Global Lipidomics Approach.

‘Shotgun’, i.e. global, in-depth or comprehensive lipidomics can survey thousands of unique lipids in a single biological specimen. Disclosed herein is a lipidomics approach using direct injection, electrospray ionization coupled with highly accurate mass spectrometers (ESI-MS) for cataloguing and quantifying lipids in tissue, cells or body fluids. Lipidomics can complement peptidomic and proteomic methods. Indeed, colostrum and milk samples can be readily fractionated into lipid- and protein-rich layers. Because there are serum lipid abnormalities associated with PRMDs in animals with established disease, and because colostrum and milk are both rich in lipids and readily accessible for collection, the applicant examined lipid expression differences between dairy cows later developing PRMDs and dairy cows that remain healthy, and disclose a panel of predictive biomarkers.

A global lipidomics approach has several advantages over prior methods. The disclosed methods may overcome the current limitations of biomarker testing and find early lipid biomarkers in samples that are easy to obtain and that are effective in predicting PRMDs in asymptomatic postpartum dairy cows. The disclosed biomarkers substantially outperform previously-available biomarkers, and even showed biologic consistency and redundancy.

Lipid Biomarkers in Colostrum and Milk.

The present disclosure represents the first disclosure of lipid biomarkers in dairy cattle obtained prior to appearance of PRMDs using early (e.g., day 0-1) postpartum colostrum and early (e.g., days 4-7) postpartum milk. Testing colostrum and/or milk provides several advantages over testing blood. For example, milk and colostrum are readily accessible, cheap, and cause less distress to the animal than blood sampling.

Thus, in one aspect, methods are provided for managing dairy cattle by sampling colostrum or milk from a cow 0-7 days after parturition; analyzing the sample for levels of one or more lipid markers; identifying a cow at increased risk of production-related metabolic disease (PRMD) based on the one or more lipid markers; and separating the cow from the herd.

As used herein, the term “parturition” refers to the action of giving birth to offspring. As used herein, the term “postpartum” refers to the period of time following parturition. For example, postpartum day 4 refers to the day that is 4 days after parturition is completed.

The methods and biomarkers disclosed herein are useful for identifying cattle that are more likely to develop PRMDs. Thus, in one aspect, methods are provided for determining risk for production-related metabolic disease (PRMD) in cattle including obtaining a colostrum or milk sample from a post-partum cow 0-7 days after parturition; analyzing the sample for levels of one or more lipid markers; and determining if the cow is at increased risk of PRMD.

The disclosed methods are most useful for early identification of cattle at risk for developing particular PRMDs, while those cattle are still asymptomatic. While some PRMDs develop prior to or immediately around parturition, in the majority of animals day 1 colostrum results or even day 4 milk results will precede the disease by several days to a few weeks, and as such could provide valuable information regarding still asymptomatic animals at risk.

In some embodiments, the cow determined to be at increased risk of PRMD is subsequently treated. This may involve, for example, administration of a suitable medicament, therapy, isolation, rest, or nutritional intervention. Treatment in this early stage may be effective for alleviating, or preventing onset of, a PRMD.

In a one embodiment, the colostrum is sampled 0 to 24 hours after parturition. In one embodiment, the colostrum is sampled 0 to 12 hours after parturition.

In some embodiments, the colostrum-derived lipid markers are selected from the group of lipid markers having a mass to charge ratio of about 344.23, 570.46, 586.54, 652.55, 682.56, 855.75, 906.84, 919.83, and combinations thereof, as determined by electrospray injection time-of-flight mass-spectrometry (ESI-TOF MS) in ammonium acetate.

In embodiments, the colostrum-derived lipid marker has a mass to charge ratio of about 344.23. In embodiments, the colostrum-derived lipid marker has a mass to charge ratio of about 570.46. In embodiments, the colostrum-derived lipid marker has a mass to charge ratio of about 586.54. In embodiments, the colostrum-derived lipid marker has a mass to charge ratio of about 652.55. In embodiments, the colostrum-derived lipid marker has a mass to charge ratio of about 682.56. In embodiments, the colostrum-derived lipid marker has a mass to charge ratio of about 855.75. In embodiments, the colostrum-derived lipid marker has a mass to charge ratio of about 906.84. In embodiments, the colostrum-derived lipid marker has a mass to charge ratio of about 919.83.

In some embodiments, any one, two, three, four, five, six, seven, or eight of the disclosed lipid markers, based on mass to charge ratio, are analyzed.

As used herein, and with respect to the mass to charge ratio of lipid markers in various embodiments disclosed herein, the term “about”—unless otherwise specified—means a mass to charge ratio that reflects the indicated value +/−0.03. For example, it is understood that “about 570.46” shall mean “570.46+/−0.03”, including all values between 570.43 and 570.49, inclusive.

In some embodiments, the colostrum-derived lipid markers have an elemental composition as observed in actual mass spectra as selected from C₄₀H₅₆O+NH₄⁺, C₃₅H₆₈O₅+NH₄⁺, C₅₅H₉₈O₆+H⁺, C₄₁H₇₆O₆+Na₄⁺, C₂₁H₂₆O₃+Na₄⁺, C₄₀H₇₄O₅+NH₄⁺, C₅₈H₁₁₀O₆+0H, and combinations thereof, in the ammonium acetate adduct form.

In embodiments, the colostrum-derived lipid marker has an elemental composition of C₄₀H₅₆O+NH₄⁺. In embodiments, the colostrum-derived lipid marker has an elemental composition of C₃₅H₆₈O₅+NH₄⁺. In embodiments, the colostrum-derived lipid marker has an elemental composition of C₅₅H₉₈O₆+H⁺. In embodiments, the colostrum-derived lipid marker has an elemental composition of C₄₁H₇₆O₆+NH₄⁺. In embodiments, the colostrum-derived lipid marker has an elemental composition of C₂₁H₂₆O₃+NH₄⁺. In embodiments, the colostrum-derived lipid marker has an elemental composition of C₄₀H₇₄O₅+NH₄⁺. In embodiments, the colostrum-derived lipid marker has an elemental composition of C₅₈H₁₁₀O₆+OH.

In some embodiments, any one, two, three, four, five, six, or seven of the disclosed lipid markers, based on elemental compositions, are analyzed.

In some embodiments, the lipid markers include DG (16:0/16:0), TG (16:0/18:1/18:3), DG (18:2/19:0), an oxidized TG (18:0/18:0/19:1)+OH, an oxidized TG (18:0/18:1/19:0)+OH, and combinations thereof.

In embodiments, the lipid marker is DG (16:0/16:0). In embodiments, the lipid marker is TG (16:0/18:1/18:3). In embodiments, the lipid marker is DG (18:2/19:0). In embodiments, the lipid marker is an oxidized TG (18:0/18:0/19:1)+OH. In embodiments, the lipid marker is an oxidized TG (18:0/18:1/19:0)+OH.

In some embodiments, any one, two, three, four, or five of the disclosed colosrum-derived lipid markers, based on the indicated structures, are analyzed.

In one embodiment, the milk is sampled 4-7 days after parturition. In some embodiments, the milk is sampled 4 days after parturition. In some embodiments, the milk is sampled 5 days after parturition. In some embodiments, the milk is sampled 6 days after parturition. In some embodiments, the milk is sampled 7 days after parturition.

In some embodiments, the milk-derived lipid markers are selected from the group of lipid markers having a mass to charge ratio of about 642.56, 652.55, 740.67, 906.84, 919.83, and combinations thereof, as determined by ESI-TOF MS.

In embodiments, the milk-derived lipid marker has a mass to charge ratio of about 642.56. In embodiments, the milk-derived lipid marker has a mass to charge ratio of about 652.55. In embodiments, the milk-derived lipid marker has a mass to charge ratio of about 740.67. In embodiments, the milk-derived lipid marker has a mass to charge ratio of about 906.84. In embodiments, the milk-derived lipid marker has a mass to charge ratio of about 919.83.

In some embodiments, any one, two, three, four, or five of the disclosed milk-derived lipid markers, based on mass to charge ratio, are analyzed.

In some embodiments, the milk-derived lipid markers have an elemental composition as observed in actual mass spectra as selected from C₃₉H₇₆O₅+Na₄⁺, C₅₇H₁₀₈O₆+NH₄⁺, C₄₅H₈₆O₆+NH₄⁺, C₅₈H₁₁₀O₆+OH, C₄₀H₇₄O₅+NH₄⁺, and combinations thereof.

In embodiments, the milk-derived lipid marker has an elemental composition of C₃₉H₇₆O₅+NH₄⁺. In embodiments, the milk-derived lipid marker has an elemental composition of C₅₇H₁₀₈O₆+NH₄⁺. In embodiments, the milk-derived lipid marker has an elemental composition of C₄₅H₈₆O₆+NH₄⁺. In embodiments, the milk-derived lipid marker has an elemental composition of C₅₈H₁₁₀O₆+OH. In embodiments, the milk-derived lipid marker has an elemental composition of C₄₀H₇₄O₅+Na₄⁺.

In some embodiments, any one, two, three, four, or five of the disclosed milk-derived lipid markers, based on elemental compositions, are analyzed.

In embodiments, the milk-derived lipid markers are selected from DG (18:0/18:0), TG (18:0/18:0/18:1), TG (12:0/14:0/16:0), an oxidized TG (18:0/18:0/19:1)+OH, an oxidized TG (18:0/18:1/19:0)+OH, DG (18:2/19:0), and combinations thereof.

In embodiments, the milk-derived lipid marker is DG (18:0/18:0). In embodiments, the milk-derived lipid marker is TG (18:0/18:0/18:1). In embodiments, the milk-derived lipid marker is TG (12:0/14:0/16:0). In embodiments, the milk-derived lipid marker is TG (12:0/14:0/16:0). In embodiments, the milk-derived lipid marker is an oxidized TG (18:0/18:1/19:0)+OH. In embodiments, the milk-derived lipid marker is DG (18:2/19:0).

In some embodiments, any one, two, three, four, five, or six of the disclosed milk-derived lipid markers, based on the indicated structures, are analyzed.

The methods and lipid markers disclosed herein may be associated with various PRMDs, including but not limited to, hypocalcemia, fatty liver syndrome, ketosis, retained placenta, obturator nerve paresis, and displaced abomasum. In one embodiment, the PRMD is hypocalcemia. In one embodiment, the PRMD is fatty liver syndrome. In one embodiment, the PRMD is ketosis. In one embodiment, the PRMD is retained placenta. In one embodiment, the PRMD is obturator nerve paresis. In one embodiment, the PRMD is displaced abomasum.

In some embodiments, cows with elevated levels of colostrum-associated lipid markers having a mass to charge ratio of about 344.23, 570.46, 682.56, 855.75, and combinations thereof, as compared to levels in normal controls, have an increased risk of PRMD's. Thus, in some embodiments, a cow with elevated levels of any one, two, three, or four of the indicated lipid markers, based on mass to charge ratio, have an increased risk of PRMDs.

In some embodiments, cows with decreased levels of a colostrum-associated lipid marker having a mass to charge ratio of about 586.54, as compared to levels in normal controls, have an increased risk of PRMD's.

In some embodiments, cows with decreased levels of a milk-associated lipid marker having a mass to charge ratio of about 642.56 and/or 740.67, as compared to levels in normal controls, have an increased risk of PRMD's.

In some embodiments, cows with increased levels of a milk-associated lipid marker having a mass to charge ratio of about 906.84, as compared to levels in normal controls, have an increased risk of PRMD's.

### PRMD Lipid Biomarker Panels:

In one aspect, a panel of lipid markers of PRMD in milk or colostrum of post-parturient dairy cattle is disclosed.

Colostrum-specific, milk-specific, and mixed panels of lipid markers optimized for their ability to classify at-risk and healthy animals may be useful biomarkers for later routine application. The lipid markers disclosed herein show predictive abilities with greater than 75% sensitivity and specificity. The disclosed panel of lipid markers may also be used to develop cutoffs or a specific risk index, i.e., a numeric likelihood of developing PRMDs based on specific quantities of the several biomarkers as part of one or more panels. Thus, in some embodiments, a single collection of colostrum (and potentially milk) with lipidomic measurement of targeted lipids may be used to determine the percent likelihood of an animal developing a PRMD. The marked alterations in TGs (e.g., in milk) provides a previously unrecognized change that may yield important insights into PRMD pathology.

In some embodiments, the panel includes one or more lipid markers selected from the group having a mass to charge ratio of about 344.23, 570.46, 586.54, 642.56, 652.55, 682.56, 740.67, 855.75, 906.84, 919.83, and combinations thereof, as determined by electrospray injection time-of-flight mass-spectrometry (ESI-TOF MS).

In embodiments, the panel includes a lipid marker having a mass to charge ratio of about 344.23. In embodiments, the panel includes a lipid marker having a mass to charge ratio of about 570.46. In embodiments, the panel includes a lipid marker having a mass to charge ratio of about 586.54. In embodiments, the panel includes a lipid marker having a mass to charge ratio of about 642.56. In embodiments, the panel includes a lipid marker having a mass to charge ratio of about 652.55. In embodiments, the panel includes a lipid marker having a mass to charge ratio of about 682.56. In embodiments, the panel includes a lipid marker having a mass to charge ratio of about 740.67. In embodiments, the panel includes a lipid marker having a mass to charge ratio of about 855.75. In embodiments, the panel includes a lipid marker having a mass to charge ratio of about 906.84. In embodiments, the panel includes a lipid marker having a mass to charge ratio of about 919.83.

In some embodiments, the panel includes any one, two, three, four, five, six, seven, eight, nine, or ten of the disclosed lipid markers, based on mass to charge ratio.

In some embodiments, the panel includes one or more lipid markers having an elemental composition as observed in MS that is selected from the group consisting of: C₄₀H₅₆O+NH₄⁺, C₃₅H₆₈O₅+NH₄⁺, C₅₅H₉₈O₆+H⁺, C₃₉H₇₆O₅+NH₄⁺, C₅₇H₁₀₈O₆+NH₄⁺, C₄₁H₇₆O₆+NH₄⁺, C₂₁H₂₆O₃+NH₄⁺, C₅₈H₁₁₀O₆+OH, C₄₀H₇₄O₅+NH₄⁺, C₄₅H₈₆O₆+NH₄⁺, and combinations thereof.

In embodiments, the panel includes the lipid marker having the elemental composition C₄₀H₅₆O+NH₄⁺. In embodiments, the panel includes the lipid marker having the elemental composition C₃₅H₆₈O₅+NH₄⁺. In embodiments, the panel includes the lipid marker having the elemental composition C₅₅H₉₈O₆+H⁺. In embodiments, the panel includes the lipid marker having the elemental composition C₃₉H₇₆O₅+NH₄⁺. In embodiments, the panel includes the lipid marker having the elemental composition C₅₇H₁₀₈O₆+NH₄⁺. In embodiments, the panel includes the lipid marker having the elemental composition C₄₁H₇₆O₆+NH₄⁺. In embodiments, the panel includes the lipid marker having the elemental composition C₂₁H₂₆O₃+NH₄⁺. In embodiments, the panel includes the lipid marker having the elemental composition C₅₈H₁₁₀O₆+OH. In embodiments, the panel includes the lipid marker having the elemental composition C₄₀H₇₄O₅+NH₄⁺. In embodiments, the panel includes the lipid marker having the elemental composition C₄₅H₈₆O₆+NH₄⁺.

In some embodiments, the panel includes any one, two, three, four, five, six, seven, eight, nine, or ten of the disclosed lipid markers, based on elemental composition.

In some embodiments, the panel includes lipid markers selected from the group consisting of DG (16:0/16:0), TG (16:0/18:1/18:3), DG (18:2/19:0), oxidized TG (18:0/18:0/19:1)+OH, oxidized TG (18:0/18:1/19:0)+OH, DG (18:0/18:0), TG (18:0/18:0/18:1), TG(12:0/14:0/16:0), TG (18:0/18:0/19:1)+OH, TG (18:0/18:1/19:0)+OH, and combinations thereof.

In embodiments, the panel includes DG (16:0/16:0). In embodiments, the panel includes TG (16:0/18:1/18:3). In embodiments, the panel includes DG (18:2/19:0). In embodiments, the panel includes the oxidized TG (18:0/18:0/19:1)+OH. In embodiments, the panel includes the oxidized TG (18:0/18:1/19:0)+OH. In embodiments, the panel includes DG (18:0/18:0). In embodiments, the panel includes TG (18:0/18:0/18:1). In embodiments, the panel includes TG(12:0/14:0/16:0). In embodiments, the panel includes TG (18:0/18:0/19:1)+OH. In embodiments, the panel includes TG (18:0/18:1/19:0)+OH.

In some embodiments, the panel includes any one, two, three, four, five, six, seven, eight, nine, or ten of the disclosed lipid markers, based on the indicated structures.

Markers found in colostrum have the advantage of being available at an earlier time point with greater opportunity for intervention in at-risk animals. The disclosed colostrum biomarker panel provides greater than 90.0% sensitivity. Though not expressed as early as colostrum, milk markers may likewise still be predictive of animals at risk for PRMD development after day 4. Identifying changes in the lipid composition in colostrum and additionally milk might reveal altered metabolism that underlies and potentially contributes to PRMDs. The disclosed milk biomarker panel provides a sensitivity of greater than 75.0%.

Analyzing colostrum and milk from the same animals may allow for an optimized combined colostrum and milk biomarker panel. Thus, in some embodiments, a PRMD biomarker panel includes multiple lipid markers from a colostrum data set, a milk data set, and/or a combined lipid marker data set (lipid markers showing significant changes between control and PRMD groups in both colostrum and milk). A combined lipid marker panel may be associated with greater sensitivity (e.g., greater than 85%, 86%, 87%, 88%, 89%, 90%, 91%, 92%, 93%, 94%, 95%, 96%, 97%, 98%, or 99%), as compared to a panel with a single lipid marker or a relatively small number of markers.

Analysis of Lipid Profiles in Colostrum and Milk.

In one aspect, methods are provided for analyzing lipid profiles in cattle, including obtaining a colostrum or milk sample from a post-partum cow 0-7 days after parturition; extracting lipids from the sample; and analyzing the sample using mass-spectrometry for levels of one or more lipid markers. In some embodiments, the sample is analyzed using electrospray injection time-of-flight mass-spectrometry (ESI-TOF MS).

The disclosure further provides novel approaches to isolation and characterization of a lipid biomarkers in milk and colostrum.

ESI-MS lipid characterization has been used for other medical indications (Jiang et al. 2000; Kim et al. 2009). In some embodiments, the disclosed methods include the step of diluting the original organic lipid extract (e.g., 500 times) before ESI-MS to allow the great majority of the observed lipids to fall within the linear dynamic concentration range of the instrument, i.e. the range over which ion signal is directly proportional to the analyte concentration.

Using the disclosed methods to analyze colostrum and milk samples, the applicants were able to identify a total of 1800-2000 different ion peaks using the positive ion mode alone (abundance >600 ion count) in one study.

Statistical linear discriminative analysis can be used to combine and assess the performance of sets or panels of discriminating biomarkers previously. The results of such modeling provide promising biomarker signatures for colostrum, for milk and for a combination of milk, colostrum and “shared” biomarkers, i.e. biomarkers that were significantly different in both biological specimens. Predictive sensitivities and specificities were in general at or above 80%. The combined set was able to completely discriminate between asymptomatic animals developing PRMDs later and animals that remained healthy.

### Lipid Biomarkers as Indicators of Metabolic Pathway Disruption

Reduced levels of shorter chain fatty acid TGs seen in animals destined to develop PRMDs may suggest that those cows have an inadequate ability to generate their own TGs when they have to meet high production demands after the onset of lactation. These global differences in classes of related lipids are remarkably consistent and to some degree uniform (i.e. several similar lipids all part of the same class changed in concert) suggesting marked differences in milk biology between animals that will develop PRMDs and those that remain healthy, and a clear harbinger of later PRMDs.

Surprisingly, the applicants identified a different trend in colostrum. Statistically significant biomarkers consisting of both short chain and longer chain TGs are elevated for 55 out of 61 TG species in the colostrum of asymptomatic animals later developing PRMDs. Without wishing to be bound to any particular theory, colostrum production and milk production are representative of different stages in lactation and also somewhat different underlying biology. Colostrum secreted within 24 hours after calving has a distinctive fatty composition compared with the secretion produced on day 4 after calving in dairy cows. Nevertheless, in colostrum, all lipid biomarkers representative of both short chain and long chain fatty acid TGs were significantly increased in those animals that later developed PRMDs. Given the timing of colostrum versus milk production, the data strongly suggests that there are preexisting metabolic problems prior to calving in those animals that later develop PRMDs that lead to increased TGs in colostrum, and that the demands of milk production as early as day 4 postpartum deplete endogenously produced TGs quickly in animals destined to develop PRMDs. Collectively, the data suggest that there are profoundly compromised metabolic pathways that lead to or reflect PRMDs, but these remain to be fully identified.

Thus, colostrum and day four milk have significantly altered lipid compositions that predate and predict the development of PRMDs in most affected dairy cows. Some of these differences suggest marked changes in lipid biosynthesis as part of the run up to PRMDs. The optimized panels of these biomarkers show promise as a means to identify most of the dairy cows developing PRMDs early enough to allow for dietary or other interventions to preserve these animals.

### STATEMENTS

The following statements present various additional embodiments of the disclosure:

Statement 1: A method for managing dairy cattle comprising sampling colostrum or milk from a cow 0-7 days after parturition; analyzing the sample for levels of one or more lipid markers; identifying a cow at increased risk of production-related metabolic disease (PRMD) based on the one or more lipid markers; and separating the cow.

Statement 2: A method of analyzing lipid profiles in cattle comprising obtaining a colostrum or milk sample from a post-partum cow 0-7 days after parturition; extracting lipids from the sample; and analyzing the sample using electrospray injection time-of-flight mass-spectrometry (ESI-TOF MS) for levels of one or more lipid markers.

Statement 3: The method of statement 1 or 2, wherein the colostrum is sampled 0 to 24 hours after parturition.

Statement 4: The method of any one of statements 1, 2, and 3, wherein the colostrum is sampled 0 to 12 hours after parturition.

Statement 5: The method of any one of statements 1, 2, 3, and 4, wherein the lipid markers are selected from the group of lipid markers having a mass to charge ratio of about 344.23, 570.46, 586.54, 652.55, 682.56, 855.75, 919.83, and combinations thereof, as determined by electrospray injection time-of-flight mass-spectrometry (ESI-TOF MS) in ammonium acetate.

Statement 6: The method of any one of statements 1, 2, 3, 4, and 5, wherein the lipid markers have an elemental composition selected from the group consisting of: C₄₀H₅₆O+NH₄⁺, C₃₅H₆₈O₅+NH₄⁺, C₅₅H₉₈O₆+H⁺, C₄₁H₇₆O₆+NH₄⁺, C₂₁H₂₆O₃+NH₄⁺, C₄₀H₇₄O₅+NH₄⁺, C₅₈H₁₁₀O₆+OH, and combinations thereof.

Statement 7: The method of any one of statements 1, 2, 3, 4, 5, and 6, wherein the lipid markers are selected from the group consisting of: DG (16:0/16:0), TG (16:0/18:1/18:3), DG(18:2/19:0), oxidized TG (18:0/18:0/19:1)+OH, oxidized TG (18:0/18:1/19:0)+OH, and combinations thereof.

Statement 8: The method of statements 1 or 2, wherein the milk is sampled 4-7 days after parturition.

Statement 9: The method of any one of statements 1, 2, and 8, wherein the lipid markers are selected from one or more lipid markers having a mass to charge ratio of about 642.56, 652.55, 740.67, 906.84, 919.83, and combinations thereof, as determined by ESI-TOF MS.

Statement 10: The method of any one of statements 1-2 and 8-9, wherein the lipid markers have an elemental composition selected from the group consisting of: C₃₉H₇₆O₅+Na₄⁺, C₅₇H₁₀₈O₆+NH₄⁺, C₄₅H₈₆O₆+NH₄⁺, C₅₈H₁₁₀O₆+OH, C₄₀H₇₄O₅+NH₄⁺, and combinations thereof.

Statement 11: The method of any one of statements 1-2 and 8-10, wherein the lipid markers are selected from the group consisting of: DG (18:0/18:0), TG (18:0/18:0/18:1), TG(12:0/14:0/16:0), TG (18:0/18:0/19:1)+OH, TG (18:0/18:1/19:0)+OH, DG(18:2/19:0), and combinations thereof.

Statement 12: The method of any one of statements 1-7, wherein cows with elevated levels of lipid markers having a mass to charge ratio of about 344.23, 570.46, 682.56, and/or 855.75, as compared to levels in normal controls, have an increased risk of PRMD's.

Statement 13: The method of any one of statements 1-7 and 12, wherein cows with decreased levels of a lipid marker having a mass to charge ratio of about 586.54, as compared to levels in normal controls, have an increased risk of PRMD's.

Statement 14: The method of any one of statements 1-2 and 8-11, wherein cows with decreased levels of a lipid marker having a mass to charge ratio of about 642.56 and/or 740.67, as compared to levels in normal controls, have an increased risk of PRMD's.

Statement 15: The method of any one of statements 1-2 and 8-12, wherein cows with increased levels of a lipid marker having a mass to charge ratio of about 906.84, as compared to levels in normal controls, have an increased risk of PRMD's.

Statement 16: A panel of lipid markers of production-related metabolic disease in milk or colostrum from 0-7 days post-parturient cattle, the panel comprising one or more lipid markers selected from the group having a mass to charge ratio of about: 344.23, 570.46, 586.54, 642.56, 652.55, 682.56, 740.67, 855.75, 906.84, 919.83, and combinations thereof, as determined by electrospray injection time-of-flight mass-spectrometry (ESI-TOF MS).

Statement 17: The panel of lipid markers of statement 16, wherein the lipid markers have an elemental composition selected from the group consisting of: C₄₀H₅₆O+NH₄⁺, C₃₅H₆₈O₅+NH₄⁺, C₅₅H₉₈O₆+H⁺, C₃₉H₇₆O₅+NH₄⁺ and C₅₇H₁₀₈O₆+NH₄⁺, C₄₁H₇₆O₆+NH₄⁺, C₂₁H₂₆O₃+NH₄⁺, C₅₇H₁₀₈O₆+NH₄⁺, C₅₈H₁₁₀O₆+OH, C₄₀H₇₄O₅+NH₄⁺, C₄₅H₈₆O₆+NH₄⁺, and combinations thereof.

Statement 18: The panel of lipid markers of statements 16 or 17, wherein the lipid markers are selected from the group consisting of: DG (16:0/16:0), TG (16:0/18:1/18:3), DG(18:2/19:0), oxidized TG (18:0/18:0/19:1)+OH, oxidized TG (18:0/18:1/19:0)+OH, DG (18:0/18:0), TG (18:0/18:0/18:1), TG(12:0/14:0/16:0), TG (18:0/18:0/19:1)+OH, TG (18:0/18:1/19:0)+OH, and combinations thereof.

Statement 19: The panel of lipid markers of any one of statements 16-18, wherein the lipid markers are present in milk 4-7 days after parturition.

Statement 20: The panel of lipid markers of any one of statements 16-18, wherein the lipid markers are present in colostrum 0 to 24 hours after parturition.

Statement 21: The method of any one of statements 1-15, wherein the PRMD is selected from the group consisting of: hypocalcemia, fatty liver syndrome, ketosis, retained placenta, obturator nerve paresis (OP), and displaced abomasum.

Statement 22: The panel of lipid markers of any one of statements 16-20, wherein the PRMD is selected from the group consisting of: hypocalcemia, fatty liver syndrome, ketosis, retained placenta, obturator nerve paresis (OP), and displaced abomasum.

### Example

The applicants conducted a nested, case-controlled discovery study to identify lipid biomarkers in colostrum (CS) and milk (MK) in asymptomatic, early postpartum dairy cows as predictors of PRMD. The study employed a comprehensive, global lipidomics approach measuring hundreds of previously unmeasured lipids to find those present in significantly altered concentrations in PRMD versus normal postpartum specimens. A large set of quantitatively-different candidate markers was reduced to more highly predictive sets comprised of a few markers (<10), thereby generating hypothetically important PRMD predictive sets for further evaluation. These biomarkers can be used as a cost-effective, non-invasive tool to determine PRMD resistance or risk, and as an aid in cattle management.

### Methods:

Animal Population and Sample Collection.

A population of 210 clinically-normal, asymptomatic Holstein cows were prospectively enrolled, including primiparous first lactation (n=101) and multiparous (n=109) animals. After calving, animals were followed for 60 days. Heifers were about two years old and cows were 3-6 years old, with parity and lactation numbers of 3-5. Primiparous and multiparous cows with similar predicted calving dates were housed alike, in separate pens. Total mixed rations formulated for each parity were fed ad libitum, with cows consuming a dry cow diet pre-partum, and a fresh cow early-lactation diet postpartum.

PRMDs were diagnosed using standard criteria. Animals developing PRMDs were frequency matched with controls of similar calving date, age, and lactation number from the same prospective cohort. Five study animals experienced retained placenta (RP) at parturition; four of these resolved with gentle traction to assist expulsion. Although these animals produced adequate milk, they were excluded as controls and not included in the colostrum and milk lipid analysis. Four cows developed milk fever within 24 hours of calving (i.e., before samples could be collected), and were also excluded.

Composite aliquots of colostrum secreted on the day of parturition, and milk produced on the fourth day of lactation were collected from the four quarters of each cow's udder into 50 mL conical vials and frozen in ice, then flash frozen in liquid N₂ and stored at −80° C. Three cows (ID 19010, 21389 and 23567) developed PRMD or were no longer in the herd at postpartum day 4, so no milk was collected. Two cows (ID 23608 and 23971) did not have milk samples analyzed due to specimen loss or the specimen being compromised.

A total of 21 cows developed PRMDs within 1-27 days of calving. These PRMDs included ketosis, left displaced abomasum (LDA), milk fever (MF), fatty liver, and/or hind limb weakness attributed to obturator nerve paresis (OP). See Table 1. These PRMD animals were frequency matched to cows in the same cohort without medical or lactation problems to form the control group. Matching criteria were calving date, age, and lactation number (parity) to ensure that observed lipid differences were due to PRMD pathology and not feeding, season, animal age bias, or differences in care. CASE: developed PRMD; CTL: control; MF: hypocalcemia, commonly known as milk fever; LDA: left displaced abomasum; OP: obturator nerve paresis; CS: postpartum day 1 colostrum; MK: postpartum day 4 milk; PROB: probable.

TABLE 1
Animal Population Characteristics.

| Animal Status | ID | Diagnosis | Lipids Tested |
|---|---|---|---|
| CTL | 19048 | n/a | CS + MK |
| CTL | 20505 | n/a | CS + MK |
| CTL | 20804 | n/a | CS + MK |
| CTL | 20873 | n/a | CS + MK |
| CTL | 21132 | n/a | CS + MK |
| CTL | 21155 | n/a | CS |
| CTL | 21247 | n/a | CS + MK |
| CTL | 21859 | n/a | CS + MK |
| CTL | 22219 | n/a | CS + MK |
| CTL | 22598 | n/a | CS + MK |
| CTL | 22877 | n/a | CS + MK |
| CTL | 23609 | n/a | CS + MK |
| CTL | 23772 | n/a | CS + MK |
| CTL | 26566 | n/a | CS + MK |
| CTL | 26558 | n/a | CS + MK |
| CTL | 26678 | n/a | CS + MK |
| CTL | 26776 | n/a | CS + MK |
| CTL | 26852 | n/a | CS + MK |
| CTL | 26998 | n/a | CS + MK |
| CTL | 29241 | n/a | CS + MK |
| CTL | 29552 | n/a | CS + MK |
| CTL | 29554 | n/a | CS |
| CTL | 29610 | n/a | CS |
| CASE | 14112 | DIED | CS + MK |
| CASE | 16320 | MF | CS + MK |
| CASE | 17829 | OP, DIED | CS + MK |
| CASE | 17841 | MF, DIED | CS + MK |
| CASE | 19010 | MF | CS |
| CASE | 20594 | LDA | CS + MK |
| CASE | 20712 | LDA | CS + MK |
| CASE | 21389 | LDA | CS + MK |
| CASE | 22377 | LDA | CS + MK |
| CASE | 23567 | MF, LDA | CS |
| CASE | 23608 | LDA | CS |
| CASE | 23762 | LDA | CS + MK |
| CASE | 23971 | LDA | CS |
| CASE | 25249 | LDA | CS + MK |
| CASE | 25853 | LDA | CS + MK |
| CASE | 26035 | MF, OP | CS + MK |
| CASE | 26832 | LDA | CS + MK |
| CASE | 29337 | LDA | CS + MK |
| CASE | 29551 | LDA | CS + MK |
| CASE | 29685 | LDA | CS + MK |
| CASE | 23374 | PROB MF | CS + MK |

Lipid Extraction.

Colostrum and milk samples were processed for lipidomic analysis. Samples frozen at −80° C. were thawed completely at room temperature. The lipid layer and aqueous protein-rich sublayer of colostrum (or milk) were separated by centrifugation for 20 min at 650×g at 4° C. After separation, 10 mg of the upper, lipid-containing layer were mixed with 3.8 mL of a solution of 2:1:1.25 (v/v/v) chloroform:methanol:isopropanol, as described by Bligh (1959). After shaking for ˜30 sec until complete lipid dissolution, 1.2 mL of double-distilled deionized water was added and the mixture was shaken again, and allowed to sit for organic and aqueous layer separation.

Following overnight incubation at 37° C., the lower organic phase was collected for complete lipid extraction and diluted 500× with a 2:1:1.25 (v/v/v) solution of chloroform:methanol:isopropanol containing 15 mM of ammonium acetate and the lipid standard archaeol (6 nM) for instrumental analysis. Reproducibility and quantitative comparisons using direct injection lipidomics are improved by adding one or more synthetic lipid standards to the specimen to be analyzed. However, markers for the lipids under investigation had not previously been identified. Thus, a synthetic lipid standard not present in animals—archeol—was added to each specimen.

Because of the somewhat long extraction protocol, lipid stability was also assessed for both milk and colostrum biomarkers. Aliquots of the organic extract from colostrum and milk containing lipid markers were removed at 0, 8 and 24 hours and assayed in triplicate. Lipids considered potential biomarkers were evaluated by mass spectrometry as part of a single run. Multiple runs of the same biologic specimen were also analyzed for quantitative reproducibility of the biomarkers of interest without normalization to internal standards, to determine relative standard deviation as an estimate of measurement reproducibility.

All candidate lipid biomarkers were determined to be completely stable during the extraction step, i.e. their abundances did not decrease (<5%) as a function of time for up to 24 hours. MS reproducibility, as suggested by relative standard deviation (RSD), of colostrum biomarkers without normalization was 17.4%. For milk biomarkers, the RSD of replicates was 11.8%.

Lipid Analysis.

Extracts were analyzed by a global lipidomics approach employing electrospray injection time-of-flight mass-spectrometry (TOF-MS). Mass spectrometric analysis was initiated by direct injection through an electrospray ionization (ESI) needle into a time-of-flight mass spectrometer (6230 ESI-TOF-MS, Agilent Technologies, Santa Clara, Calif.). The ionization voltage was set to 3.5 kV, gas pressure to 15 psi and the source was controlled by instrument software (MassHunter Workstation Data Acquisition software, Agilent). All lipid samples were infused at a flow rate of 10 μL/min by syringe pump (New Era Pump Systems, Farmingdale, N.Y., USA) and analyzed in the positive ion mode for 3 min, with MS data collected over the range of m/z 100 to 1500. Technical replicates (n=2) were performed for each sample and values averaged.

Addition of ammonium acetate to the extracts facilitates formation of charged adducts with otherwise neutral compounds. Thus, m/z peaks obtained using the foregoing ESI-TOF-MS methods reflected either the ammonium (NH₄⁺) or prononated (H⁺) form of the respective species, depending on the species present and acidity.

Data Analysis.

Instrument software (MassHunter Qualitative Analysis B.07.00 software, Agilent) was used to generate a peak list with the abundance of each lipid (in ion counts) recorded for each peak for each specimen. Two data columns were generated for each file that included the m/z values and their corresponding abundances. The peaks were aligned between each run according to the instrument-assigned m/z values and the associated abundances were recorded and confirmed manually. The intensity of the lipid standard in each run was determined and used for data normalization, with the normalized abundances of the two replicates averaged.

Peaks were analyzed statistically, and species that were quantitatively different between the two groups (healthy and PRMD) were modeled to develop PRMD-predictive biomarker panels. A two-tailed Student's t-test was carried out on the normalized abundances for each group. Colostrum and milk samples were analyzed separately. Some candidate species were significantly different in both colostrum and milk. These were referred to as ‘shared’ markers. Furthermore, for these shared markers, quantitative differences between the normalized abundances for each peak were calculated by subtraction (the quantity in colostrum minus the quantity in milk), and the differences further considered in the statistical analyses.

Candidate lipid biomarkers (i.e., those having significantly different quantitative abundances between the two groups) were submitted to linear discriminative analysis to model combinations or panels of milk and/or colostrum biomarkers (SAS 9.3, SAS Institute Inc., Cary, N.C., USA), and optimized for area under the curve. Linear discriminant analysis was conducted as described by Yi et al (2008). A significance level of p<0.05 was considered significant for all tests. Discriminant analysis, grouping variable HSC, was performed for each colostrum (CS) and milk (MK) biomarker, including ‘shared’ markers.

Additionally, after reviewing the results in milk, it was observed that all lipid biomarkers of a particular mass to charge (m/z) range, specifically 572.5 to 810.7, very likely representing a common lipid type, were increased in milk of animals that later developed PRMDs, whereas all the lipid biomarkers in a higher m/z range between m/z 848 to 920, also likely representing a common lipid type, were reduced in the animals later developing PRMDs. To test the significance of this finding, a 2×2 contingency table was created that looked at the classification of diseased animals with the consistency of an elevated or decreased abundance observed for lipid biomarkers based on their m/z category. A Fisher Exact test was performed and a p-value <0.05 was considered significant.

Lipid Identification.

Targeted MS/MS was applied to extracts in an effort to identify or substantially characterize each useful biomarker. A combination of “exact” mass studies, together with MS/MS fragmentation studies using collisionally-induced dissociation, was used to chemically characterize candidate biomarkers. Fragmentation data was acquired on both a QSTAR Pulsar I quadrupole orthogonal time-of-flight mass spectrometer through an IonSpray Source (Applied Biosystems, Foster City, Calif., USA) and on an Agilent 6530 accurate-mass quadrupole/time-of flight mass spectrometry (Agilent Technologies, Santa Clara, Calif., USA). Specific colostrum and milk samples were selected for characterization based on the higher abundance of the targeted lipid of interest. Samples were extracted using 2:1:1.25 (v/v/v) chloroform:methanol:isopropanol with 15 mM ammonium acetate.

Chromatogram traces were obtained from ESI total ion chromatograph (TIC) at a MS/MS level using an instrument based software program (MassHunter Qualitative Analysis B.07.00, Agilent). The exported product ion mode was broadly used for lipid identification. Predicted identities of target lipids were searched using the on-line reference site LIPID MAPS and the Elemental Composition Calculator programmed by Frank Antolasic (School of Applied Sciences, RMIT University, Melbourne, Victoria, Australia), in conjunction with the experimentally determined accurate lipid mass after determining the adduct present.

Fragmentation information was further manually evaluated in product ion mode through review of neutral loss species, or scanned fragment information. The LIPID MAPS MS fragment prediction tool (http://www.lipidmaps.org/tools/index.html) was also applied to determine predicted product ion peak lists, which often represented sn1 and sn2 acyl losses mainly for glycerolipids.

Results:

A total of 1800-2000 ion peaks, representing approximately that number of different lipids, were listed from MS runs of separately analyzed colostrum and milk samples. Peak abundances were normalized and statistically tested for apparent quantitative differences between the comparison groups.

Candidate markers with statistically different abundances (p-value <0.05) between cows that went on to develop PRMDs and their frequency-matched controls are listed in Table 2 (colostrum) and Table 3 (milk). In total, 61 statistically significant, quantitatively different lipids were discovered in colostrum, and 77 significant, quantitatively different lipids were discovered in milk. Means of data in Tables 2 and 3 were normalized to endogenous controls.

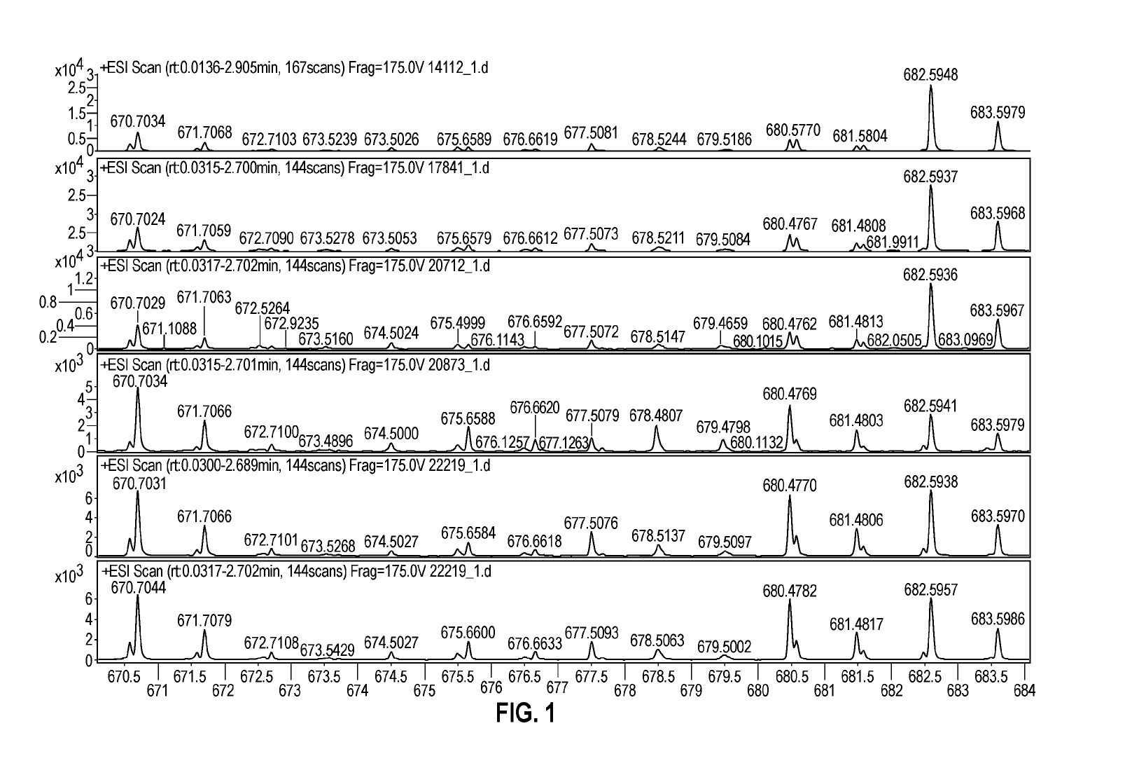

FIG. 1 shows an example for one candidate quantification using lipid profiling via TOF with representative mass spectra of lipid marker at m/z 682.59 from 6 colostrum samples. The peak at 670.70 m/z is the ammoniated internal standard archaeol (2,3-diphytanyl-sn-glycerol). The upper three samples are cows (14112, 17841, 20712) that developed PRMDs, and the bottom three are matched control cows (20873, 22219, 21859) that remained healthy. From top to bottom the abundances for m/z 682.56 were 2.6×10⁴, 1.8×10⁴, 1.1×10⁴ for later PRMD and 2.9×10³, 6.5×10³, 6.0×10³ in unaffected animals. Note variable y-axis scale.

As observed in these 6 colostrum samples' mass spectra, m/z 682.59 is more highly expressed in cows which later developed PRMDs compared to control animals that remained healthy.

TABLE 2
PRMD predictive colostrum lipid
biomarkers comparing animals with later
PRMD to resistant dairy cows.

| Marker (m/z) | Mean CTL | Mean Cases | Higher In | P-Value |
|---|---|---|---|---|
| 344.22 | 0.104 | 0.115 | CASES | 0.042 |
| 388.25 | 0.113 | 0.125 | CASES | 0.048 |
| 489.10 | 0.291 | 0.374 | CASES | 0.049 |
| 570.46 | 0.112 | 0.176 | CASES | 0.013 |
| 586.54 | 0.266 | 0.203 | CTL | 0.0014 |
| 598.50 | 0.081 | 0.113 | CASES | 0.029 |
| 612.55 | 0.078 | 0.066 | CTL | 0.044 |
| 615.56 | 0.088 | 0.069 | CTL | 0.043 |
| 648.46 | 0.115 | 0.085 | CTL | 0.049 |
| 652.55 | 0.074 | 0.098 | CASES | 0.047 |
| 654.56 | 0.653 | 0.939 | CASES | 0.015 |
| 659.52 | 0.143 | 0.198 | CASES | 0.0042 |
| 668.58 | 0.116 | 0.166 | CASES | 0.015 |
| 675.50 | 0.100 | 0.126 | CASES | 0.034 |
| 680.58 | 0.349 | 0.513 | CASES | 0.0042 |
| 682.59 | 1.405 | 2.591 | CASES | 0.00095 |
| 687.55 | 0.224 | 0.381 | CASES | 0.00041 |
| 694.60 | 0.068 | 0.107 | CASES | 0.0032 |
| 695.59 | 0.138 | 0.192 | CASES | 0.0077 |
| 696.60 | 0.148 | 0.225 | CASES | 0.0025 |
| 703.53 | 0.163 | 0.262 | CASES | 0.0014 |
| 704.55 | 0.081 | 0.134 | CASES | 0.00055 |
| 706.59 | 0.140 | 0.218 | CASES | 0.0027 |
| 708.61 | 0.366 | 0.732 | CASES | 0.00047 |
| 710.63 | 0.753 | 1.198 | CASES | 0.0028 |
| 723.62 | 0.243 | 0.400 | CASES | 0.0011 |
| 731.56 | 0.085 | 0.128 | CASES | 0.0019 |
| 732.57 | 0.082 | 0.113 | CASES | 0.0055 |
| 734.61 | 0.078 | 0.098 | CASES | 0.040 |
| 736.64 | 0.168 | 0.250 | CASES | 0.0031 |
| 749.64 | 0.068 | 0.126 | CASES | 0.00033 |
| 751.65 | 0.134 | 0.197 | CASES | 0.0030 |
| 752.66 | 0.105 | 0.139 | CASES | 0.011 |
| 848.77 | 0.750 | 0.935 | CASES | 0.017 |
| 850.78 | 2.001 | 2.587 | CASES | 0.0079 |
| 855.74 | 0.396 | 0.491 | CASES | 0.0062 |
| 858.75 | 0.075 | 0.101 | CASES | 0.00065 |
| 861.57 | 0.099 | 0.071 | CTL | 0.035 |
| 862.79 | 0.119 | 0.166 | CASES | 0.0049 |
| 864.79 | 0.263 | 0.325 | CASES | 0.0075 |
| 872.74 | 0.187 | 0.223 | CASES | 0.023 |
| 873.74 | 0.128 | 0.154 | CASES | 0.017 |
| 874.78 | 0.330 | 0.478 | CASES | 0.00041 |
| 876.80 | 0.693 | 1.366 | CASES | 0.00025 |
| 878.81 | 0.494 | 0.961 | CASES | 0.00047 |
| 881.76 | 0.175 | 0.304 | CASES | 0.000062 |
| 883.76 | 0.112 | 0.202 | CASES | 0.00011 |
| 889.79 | 0.119 | 0.143 | CASES | 0.016 |
| 890.81 | 0.111 | 0.151 | CASES | 0.0016 |
| 891.81 | 0.273 | 0.339 | CASES | 0.0074 |
| 897.73 | 0.098 | 0.158 | CASES | 0.00037 |
| 899.76 | 0.089 | 0.140 | CASES | 0.00022 |
| 900.79 | 0.139 | 0.221 | CASES | 0.00027 |
| 902.81 | 0.176 | 0.366 | CASES | 0.00045 |
| 904.82 | 0.156 | 0.333 | CASES | 0.00073 |
| 906.84 | 0.101 | 0.181 | CASES | 0.00097 |
| 917.82 | 0.099 | 0.170 | CASES | 0.00022 |
| 919.83 | 0.067 | 0.124 | CASES | 0.00015 |
| 964.59 | 0.105 | 0.073 | CTL | 0.016 |
| 965.70 | 0.064 | 0.107 | CASES | 0.00066 |
| 967.71 | 0.102 | 0.123 | CASES | 0.037 |

TABLE 3
PRMD predictive milk lipid biomarkers
comparing animals with later
PRMD to resistant dairy cows.

| Marker (m/z) | Mean CTL | Mean Cases | Higher In | P-Value |
|---|---|---|---|---|
| 572.48 | 0.489 | 0.227 | CTL | 0.000057 |
| 600.51 | 0.918 | 0426 | CTL | 0.000018 |
| 626.53 | 0.531 | 0.333 | CTL | 0.00013 |
| 633.50 | 0.266 | 0.151 | CTL | 0.0000093 |
| 640.54 | 0.134 | 0.105 | CTL | 0.0059 |
| 642.58 | 0.452 | 0.270 | CTL | 0.00000028 |
| 652.55 | 0.218 | 0.164 | CTL | 0.0014 |
| 654.26 | 2.098 | 1.573 | CTL | 0.00095 |
| 656.58 | 6.780 | 4.802 | CTL | 0.000024 |
| 657.58 | 2.549 | 1.821 | CTL | 0.000026 |
| 658.58 | 0.562 | 0.415 | CTL | 0.000045 |
| 659.52 | 0.333 | 0.271 | CTL | 0.0053 |
| 661.53 | 0.620 | 0.450 | CTL | 0.000037 |
| 668.58 | 0.390 | 0.315 | CTL | 0.0031 |
| 669.58 | 0.476 | 0.311 | CTL | 0.0000020 |
| 670.59 | 0.733 | 0.530 | CTL | 0.000011 |
| 675.50 | 0.197 | 0.142 | CTL | 0.0022 |
| 677.51 | 0.440 | 0.296 | CTL | 0.0024 |
| 684.61 | 5.376 | 4.039 | CTL | 0.00045 |
| 689.56 | 0.525 | 0.404 | CTL | 0.00042 |
| 695.49 | 0.349 | 0.289 | CTL | 0.010 |
| 697.61 | 0.974 | 0.762 | CTL | 0.00019 |
| 705.54 | 0.389 | 0.280 | CTL | 0.0082 |
| 712.64 | 1.880 | 1.282 | CTL | 0.000060 |
| 717.58 | 0.219 | 0.155 | CTL | 0.000080 |
| 721.50 | 0.234 | 0.194 | CTL | 0.048 |
| 725.63 | 0.700 | 0.552 | CTL | 0.0011 |
| 732.58 | 0.163 | 0.132 | CTL | 0.028 |
| 733.57 | 0.173 | 0.118 | CTL | 0.0020 |
| 734.62 | 0.156 | 0.116 | CTL | 0.00065 |
| 738.65 | 1.049 | 0.744 | CTL | 0.00016 |
| 740.67 | 0.834 | 0.493 | CTL | 0.00037 |
| 752.66 | 0.274 | 0.230 | CTL | 0.0075 |
| 753.66 | 0.266 | 0.190 | CTL | 0.000055 |
| 754.67 | 0.185 | 0.123 | CTL | 0.000015 |
| 764.67 | 0.302 | 0.234 | CTL | 0.00075 |
| 766.69 | 0.723 | 0.478 | CTL | 0.00016 |
| 768.70 | 0.590 | 0.396 | CTL | 0.020 |
| 780.69 | 0.133 | 0.094 | CTL | 0.000026 |
| 792.70 | 0.282 | 0.221 | CTL | 0.0016 |
| 794.72 | 0.701 | 0.538 | CTL | 0.019 |
| 808.73 | 0.145 | 0.112 | CTL | 0.00046 |
| 809.74 | 0.126 | 0.096 | CTL | 0.014 |
| 810.74 | 0.140 | 0.112 | CTL | 0.028 |
| 848.76 | 0.971 | 1.206 | CASES | 0.0015 |
| 850.78 | 2.310 | 3.140 | CASES | 0.000026 |
| 855.74 | 0.353 | 0.459 | CASES | 0.0000069 |
| 862.78 | 0.206 | 0.262 | CASES | 0.0036 |
| 863.78 | 0.229 | 0.262 | CASES | 0.0056 |
| 864.79 | 0.344 | 0.445 | CASES | 0.00031 |
| 865.79 | 0.232 | 0.288 | CASES | 0.00024 |
| 872.79 | 0.204 | 0.241 | CASES | 0.025 |
| 874.78 | 0.602 | 0.884 | CASES | 0.00031 |
| 876.80 | 2.283 | 3.975 | CASES | 0.000045 |
| 878.81 | 1.794 | 2.900 | CASES | 0.000020 |
| 879.81 | 0.894 | 1.398 | CASES | 0.000021 |
| 881.76 | 0.382 | 0.623 | CASES | 0.0000060 |
| 883.76 | 0.286 | 0.430 | CASES | 0.0000074 |
| 888.79 | 0.087 | 0.123 | CASES | 0.00085 |
| 889.79 | 0.144 | 0.193 | CASES | 0.00024 |
| 890.81 | 0.213 | 0.318 | CASES | 0.00015 |
| 891.81 | 0.313 | 0.453 | CASES | 0.000013 |
| 896.75 | 0.075 | 0.090 | CASES | 0.023 |
| 897.74 | 0.223 | 0.303 | CASES | 0.013 |
| 898.76 | 0.176 | 0.233 | CASES | 0.0063 |
| 900.79 | 0.269 | 0.393 | CASES | 0.00029 |
| 902.81 | 0.637 | 1.199 | CASES | 0.000022 |
| 904.52 | 0.720 | 1.308 | CASES | 0.000013 |
| 906.84 | 0.365 | 0.588 | CASES | 0.000019 |
| 908.80 | 0.111 | 0.157 | CASES | 0.000086 |
| 909.78 | 0.133 | 0.222 | CASES | 0.0000056 |
| 915.80 | 0.075 | 0.108 | CASES | 0.00033 |
| 917.82 | 0.223 | 0.375 | CASES | 0.000056 |
| 919.83 | 0.188 | 0.292 | CASES | 0.000049 |
| 939.68 | 0.241 | 0.188 | CTL | 0.00016 |
| 967.71 | 0.197 | 0.168 | CTL | 0.0060 |

Certain classes of lipids, e.g. TGs with shorter chain fatty acids, C₁₆ and shorter, consistently changed in PRMD specimens, suggesting profound pre-PRMD pathology.

Of the lipids identified in Tables 2 and 3 above, 31 were found to be significantly different in both colostrum and milk. For these 31 ‘shared’ markers, differences between colostrum and milk values of the same peak were calculated by using the normalized abundance of that lipid in colostrum minus its normalized abundance in milk (Table 4). The differences of shared biomarkers shown in Table 4 were calculated by using the normalized abundance of colostrum minus the normalized abundance of milk. Mean CTL: mean differences of controls; Mean CASES: mean differences of animals with disease; 31 shared peaks were found as shown, p-values in the rightmost column were obtained through SAS 9.3.

TABLE 4
Statistical analysis of ‘shared’ peak differences, i.e. for lipids significantly
different between diseased and control animals in both colostrum and milk.

| Marker (m/z) | Mean CTL | Mean Cases | Difference | P-Value |
|---|---|---|---|---|
| 652.55 | −0.145 | −0.0636 | −0.081 + 0.016 | <0.0001 |
| 654.56 | −1.45 | −0.612 | −0.841 + 0.148 | <0.0001 |
| 659.52 | −0.191 | −0.067 | −0.124 + 0.024 | <0.0001 |
| 668.58 | −0.275 | −0.145 | −0.130 + 0.028 | <0.0001 |
| 675.50 | −0.0973 | −0.0107 | −0.087 + 0.021 | 0.0003 |
| 695.59 | −0.212 | −0.0903 | −0.122 + 0.023 | <0.0001 |
| 732.57 | −0.080 | −0.014 | −0.066 + 0.018 | 0.0009 |
| 734.61 | −0.078 | −0.016 | −0.062 + 0.014 | 0.0001 |
| 752.66 | −0.170 | −0.088 | −0.082 + 0.018 | <0.0001 |
| 848.76 | −0.243 | −0.285 | 0.041 + 0.093 | 0.6585 |
| 850.78 | −0.372 | −0.600 | 0.227 + 0.266 | 0.3993 |
| 855.74 | 0.039 | 0.027 | 0.012 + 0.049 | 0.8059 |
| 862.79 | −0.091 | −0.094 | 0.002 + 0.027 | 0.9297 |
| 864.79 | −0.085 | −0.116 | 0.031 + 0.038 | 0.4263 |
| 872.75 | −0.020 | −0.019 | −0.001 + 0.026 | 0.9777 |
| 874.78 | −0.278 | −0.405 | 0.126 + 0.072 | 0.0915 |
| 876.80 | −1.60 | −2.56 | 0.961 + 0.316 | 0.0046 |
| 878.81 | −1.308 | −1.900 | 0.591 + 0.201 | 0.0058 |
| 881.76 | −0.207 | −0.299 | 0.093 + 0.043 | 0.0375 |
| 883.76 | −0.173 | −0.220 | 0.046 + 0.030 | 0.1346 |
| 889.79 | −0.027 | −0.049 | 0.022 + 0.015 | 0.1629 |
| 890.81 | −0.103 | −0.163 | 0.060 + 0.025 | 0.0210 |
| 891.81 | −0.046 | −0.112 | 0.066 + 0.036 | 0.0774 |
| 897.73 | −0.125 | −0.140 | 0.015 + 0.035 | 0.6680 |
| 900.79 | −0.131 | −0.164 | 0.033 + 0.035 | 0.3417 |
| 902.81 | −0.463 | −0.816 | 0.353 + 0.099 | 0.0011 |
| 904.82 | −0.565 | −0.961 | 0.396 + 0.099 | 0.0003 |
| 906.84 | −0.265 | −0.401 | 0.136 + 0.040 | 0.0018 |
| 917.82 | −0.126 | −0.198 | 0.072 + 0.029 | 0.0194 |
| 919.83 | −0.120 | −0.163 | 0.042 + 0.022 | 0.0614 |
| 967.71 | −0.098 | −0.044 | −0.053 + 0.013 | 0.0003 |

Those markers that were directionally opposite in colostrum and milk provided dramatic differences and very low p-values. All of the non-significant differences were for markers that were directionally the same in both colostrum and milk.

Characterization of PRMD Biomarker Candidates.

After developing these optimized predictive biomarker panels, targeted MS/MS analyses were performed to chemically characterize the 10 highly relevant lipid biomarkers obtained from the three panels. As summarized in Table 2, among the 61 statistically significant biomarker candidates in colostrum, 55 of them were present in higher concentration based on their normalized abundances in dairy cows that subsequently developed PRMDs.

Lipid marker structures were chemically characterized or identified by means of targeted tandem MS/MS analyses on QqTOF-MS instruments using collisionally-induced dissociation (CID). The markers that made up the three panels identified above were submitted for this further characterization. This represented 10 unique lipid biomarkers. Of these, 5 markers were successfully classified as triacylglycerols (TG), including m/z 855.75 which appeared to represent the protonated TG (16:0/18:1/18:3) based on 2 abundant fragments at m/z 573.49 and m/z 599.48 representing neutral losses of fatty acid constituents. The third fatty acid was predicted based on mass differences. The marker m/z 906.84 was determined to be an ammoniated TG because of a peak at [M+Na₄-17]⁺. Utilizing this same approach, marker m/z 740.67 was determined with high probability to be the triglyceride [TG (12:0/14:0/16:0)+NH₄]⁺. Characterization studies on the marker m/z 919.83 yielded two possible identifications, an oxidized TG [(18:0/18:0/19:1)+OH]⁺ or an oxidized TG [(18:0/18:1/19:0)+OH]⁺. As for the marker m/z 682.59, a NH₃ neutral loss was observed in the spectra and the exact mass of that fragment at m/z 664.56 suggested its elemental composition to be C₄₁H₇₆O₆, which indicates strongly that the marker is a TG. Three of the 10 markers were categorized as diacylglycerols (DG) through the same approach, with determinations based on the fragments and neutral loss peaks in the fragmentation spectra. The fragmentation studies were most consistent with these 3 markers being DG (16:0/16:0), DG (18:0/18:0) and DG (18:2/19:0). The elemental composition of the final two lipid markers having m/z 570.46 and m/z 344.22 were determined as [C₄₀H₅₆O+NH₄]⁺ and [C₂₁H₂₆O₃+NH₄]⁺. However, these two markers were not classified into a specific lipid group due to a lack of identifiable head groups or recognizable constituent species in the fragmentation data.

Of those markers that were part of the optimized panel, 8 out of 10 lipid biomarkers were successfully identified. Even though it was challenging to confidently determine the lipid class of colostrum biomarker m/z 344.22, based on the most likely elemental composition determined as C₂₁H₂₆O₃+NH₄⁺ predicted by exact mass studies, it would suggest that it is an oxidized lipid. This marker showed a higher normalized intensity in the colostrum of case animals compared with healthy controls.

Markers found in colostrum have the advantage of being available at an earlier time than milk or other analytes (e.g., postpartum day 1), providing a greater opportunity for intervention in at-risk animals. The best colostrum biomarker panel, as found for these data using linear discriminative analysis, contained three lipids, as summarized in Table 5. The panel provided 90.0% sensitivity at 86.4% specificity. These provided 90.0% sensitivity at 86.4% specificity. This panel predicted 19 out of 22 control cows and 18 out of 20 cows with later PRMDs. DG: diacylglycerol; TG: triacylglycerol.

TABLE 5
Identification/classification of predictive PRMD lipid markers that were
part of the optimized panel for colostrum.

| Marker | Elemental | Classification | | |
|---|---|---|---|---|
| (m/z) | composition | constituents | Abundance | P-value |
| 570.46 | C₄₀H₅₆O + NH₄⁺ | Unknown lipid class | Higher in disease | 0.013 |
| 586.54 | C₃₅H₆₈O₅ + NH₄⁺ | DG (16:0/16:0) | Higher in control | 0.0014 |
| 855.75 | C₅₅H₉₈O₆ + H⁺ | TG (16:0/18:1/18:3) | Higher in disease | 0.00065 |

Milk markers may likewise be predictive of animals at risk for PRMD development after day 4. The best milk biomarker panel contained two lipids as summarized in Table 6 that provided a sensitivity of 75.0% at a specificity of 90.0%. An optimized predictor panel of milk lipids provided 75.0% sensitivity at 90.0% specificity, by predicting 12 out of 16 cows that later developed PRMDs and 18 out of 20 cows that remained healthy that were used as controls.

TABLE 6
Identification/classification of predictive PRMD milk
biomarkers. DG: diacylglycerol; TG: triacylglycerol.

| Marker | Elemental | Classification | | |
|---|---|---|---|---|
| (m/z) | composition | (constituents) | Abundance | P Value |
| 642.56 | C₃₉H₇₆O₅ + NH₄⁺ | DG(18:0/18:0) | Higher in | 2.84E−07 |
| | | | controls | |
| 906.84 | C₅₄H₁₀₈O₆ + NH₄⁺ | TG(18:0/18:0/18:1) | Higher in | 1.86E−05 |
| | | | diseases | |

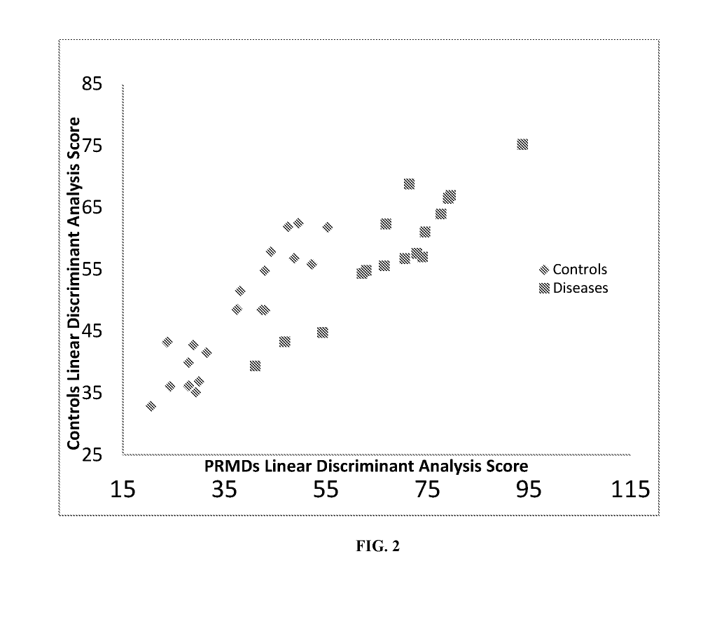

Identifying changes in the lipid composition in colostrum and milk might reveal altered metabolism that underlies and potentially contributes to PRMDs. Having colostrum and milk from the same animals allowed for an optimized combined colostrum and milk biomarker panel. This included a total of 7 lipid markers: 2 lipids from the colostrum data set, 2 lipids from the milk data set, and 3 markers represented calculated differences between colostrum and milk for the ‘shared’ marker set. This combined biomarker panel demonstrated 87.5% sensitivity at 100.0% specificity (Table 7, FIG. 2). An optimized predictor panel using a combination of colostrum, milk and the differences between milk and colostrum for ‘shared’ lipids found in both of these secretions showing significant differences in both colostrum and milk and provided 87.5% sensitivity at a specificity of 100.0% by predicting 14 out of 16 cows that later developed PRMDs and 20 out of 20 cows that remained healthy and served as controls. The difference between the normalized abundance of the biomarker for lipids with m/z 652.55, 906.84 and 919.83 indicated it was higher in milk than in colostrum.

In colostrum, the results were quite different. The statistically significant biomarkers consisting of both short chain and longer chain TGs were elevated for 55 out of 61 TG species in the colostrum of asymptomatic animals later developing PRMDs. Collectively, the data suggest that there are profoundly compromised metabolic pathways that lead to or reflect PRMDs, but these remain to be fully identified.

TABLE 7
Identification/classification of predictive PRMD biomarkers combining
colostrum (CS), milk (MK) and shared (DF, difference) markers. DG:
diacylglycerol; TG: triacylglycerol.

| Marker | Elemental | Classification | | |
|---|---|---|---|---|
| (m/z) | composition | (constituents) | Abundance | P Value |
| MK | C₃₉H₇₆O₅ + NH₄⁺ | DG(18:0/18:0) | Higher in | 2.84E-07 |
| 642.56 | | | controls | |
| CS 682.56 | C₄₁H₇₆O₆ + NH₄⁺ | TG | Higher in | 0.00095 |
| | | | diseases | |
| CS 344.23 | C₂₁H₂₆O₃ + NH₄⁺ | Unknown | Higher in | 0.0421 |
| | | lipid class | diseases | |
| DF 906.84 | C₅₄H₁₀₈O₆ + NH₄⁺ | TG(18:0/18:0/ | Higher | 0.0614 |
| | | 18:1) | changes in | |
| | | | diseases | |
| DF 919.83 | C₅₈H₁₁₀O₆ + OH | TG | Higher | 0.0018 |
| | | (18:0/18:0/ | changes in | |
| | | 19:1) + OH or | diseases | |
| | | TG | | |
| | | (18:0/18:1/ | | |
| | | 19:0) + OH | | |
| DF 652.55 | C₄₀H₇₄O₅ + NH₄⁺ | DG(18:2/19:0) | Higher | <0.0001 |
| | | | changes in | |
| | | | controls | |
| MK | C₄₅H₈₆O₆ + NH₄⁺ | TG(12:0/14:0/ | Higher in | 0.00037 |
| 740.67 | | 16:0) | controls | |

### CONCLUSIONS

Readily obtained, previously-undescribed lipid biomarkers were able to predict future PRMDs in most dairy cows days to weeks prior to clinical signs. The results also suggest marked alterations in certain lipid metabolic pathways.

The results of statistical linear discriminative analysis provided promising biomarker signatures for colostrum, for milk and for a combination of milk, colostrum and ‘shared’ biomarkers, i.e. biomarkers that were significantly different in both biological specimens. Many combinations provided useful prediction of PRMDs. Predictive sensitivities and specificities were in general at or above 80%. The combined set was able to completely discriminate between asymptomatic animals developing PRMDs later and animals that remained healthy.

FIG. 2 is a plot showing linear discriminative analysis results for the optimal set of lipid biomarkers demonstrating separation between modeled biomarker values for animals developing PRMDs later (red) and healthy unaffected animals (blue). The x-axis and y-axis represent the discriminant function scores.

A finding in the post-partum day 4 milk lipid biomarkers suggested a profound and significant change in the biological composition of triglycerides in milk between animals destined to develop PRMDs and animals that remained healthy. Among the 77 statistically significant biomarker candidates in milk listed in Table 3, those having m/z values from 572.48 to 810.74 (n=45) were all higher in controls, whereas those having m/z values from 848.76 to 919.83 (30 of 32) were higher in animals that later developed disease. The remaining 2 statistically significant biomarker candidates having m/z values of 939.68 and 967.71 were both higher in controls.

Overall, these lipid ‘class’ distinctions in milk were highly significant (p=2.3×10⁻¹⁹). All of these particular milk lipid biomarkers appear to be triglycerides (TG). Such sweeping changes in one particular subclass of compounds likely denotes substantial metabolic changes present in these asymptomatic animals that later developed PRMDs. It has been previously established that the mammary gland of the cow synthesizes de novo fatty acids with an even number of carbons ranging in chain length from 4 to 14 carbons (i.e. fatty acids 4:0 to 14:0) together with production of about half of the 16:0 fatty acids. These short fatty acids account for approximately 60% of the total fatty acids present in milk on a molar basis, respectively (Mannson et al. 2008). The remaining 40% of milk fatty acids are longer-chain (16 carbons and longer), predominantly C₁₈ fatty acids derived mainly from dietary lipids, but are also from lipolysis of adipose tissue triacylglycerols, which make their way into the circulation, mainly as plasma NEFA and triglyceride-rich lipoproteins. Thus, fatty acids incorporated into milk fat TGs are provided either from mammary de novo synthesis or from the uptake of pre-formed fatty acids from the peripheral circulation. Even though we did not determine the structure of all the milk lipid biomarkers, those lipids with m/z values from 848.76 to 919.83 almost all represent longer chain TGs based on their m/z values and on the analysis of selected lipids in this range. Indeed, those milk biomarkers that were included in the optimal panel, then fragmented and characterized having m/z values of 906.83 and m/z 919.83 were found to be TGs having longer chain fatty acid components. These were higher in clinically normal cows that later developed PRMDs (see Table 7). Milk lipids in the range of m/z 510 to 810 were very likely TGs composed of shorter chain fatty acids, based on size and selected tandem MS identifications. For example, the biomarker at m/z 740.67 was characterized as a TG having shorter chain fatty acids present, i.e., 12:0/14:0/16:0 and was found in higher levels in controls (or reduced levels in cows with later PRMDs). Because the dairy cows that developed PRMDs and the matched control group cows selected for this study were provided with the same feed, environment and care, the differences in the lipid biomarker concentrations between later affected and healthy animals cannot be attributed to diet or other environmental factor.

The long chain fatty acid C18:1 cis-9 has been previously proposed as a possible biomarker whose increased circulating concentration was broadly indicative of elevated concentrations of plasma NEFA. As for hyperketonemia, 90% of non-hyperketonemic controls showed a milk fat C18:1 cis-9-to-C15:0 ratio of 40 or lower, whereas 70% of cows suffering from hyperketonemia showed milk fat C18:1 cis-9-to-C15:0 ratios exceeding 40, which is consistent with our findings of potential different trends between longer chain TGs and shorter chain TGs. The higher expression of longer chain TGs in diseased animals may be an early compensatory response to reduced de novo maternal short chain fatty acid production by these cows.

What is claimed is:

1. A method for managing dairy cattle, the method comprising:
sampling colostrum or milk from a cow 0-7 days after parturition;
analyzing the sample for levels of one or more lipid markers;
identifying a cow at increased risk of production-related metabolic disease (PRMD) based on the one or more lipid markers; and
separating the cow.

2. The method according to claim 1, wherein the colostrum is sampled 0 to 24 hours after parturition.

3. The method according to claim 2, wherein the colostrum is sampled 0 to 12 hours after parturition.

4. The method according to claim 2, wherein the lipid markers have a mass to charge ratio selected from the group consisting of: about 344.23, 570.46, 586.54, 652.55, 682.56, 855.75, 919.83, and combinations thereof, as determined by electrospray injection time-of-flight mass-spectrometry (ESI-TOF MS) in ammonium acetate.

5. The method according to claim 2, wherein the lipid markers have an elemental composition selected from the group consisting of: C₄₀H₅₆O+NH₄⁺, C₃₅H₆₈O₅+NH₄⁺, C₅₅H₉₈O₆+H⁺, C₄₁H₇₆O₆+NH₄⁺, C₂₁H₂₆O₃+NH₄⁺, C₄₀H₇₄O₅+NH₄⁺, C₅₈H₁₁₀O₆+OH, and combinations thereof.

6. The method according to claim 2, wherein the lipid markers are selected from the group consisting of: DG (16:0/16:0), TG (16:0/18:1/18:3), DG(18:2/19:0), oxidized TG (18:0/18:0/19:1)+OH, oxidized TG (18:0/18:1/19:0)+OH, and combinations thereof.

7. The method according to claim 1, wherein the milk is sampled 4-7 days after parturition.

8. The method according to claim 7, wherein the lipid markers have a mass to charge ratio selected from the group consisting of: about 642.56, 652.55, 740.67, 906.84, 919.83, and combinations thereof, as determined by ESI-TOF MS.

9. The method according to claim 7, wherein the lipid markers have an elemental composition selected from the group consisting of: C₃₉H₇₆O₅+NH₄⁺, C₅₇H₁₀₈O₆+NH₄⁺, C₄₅H₈₆O₆+NH₄⁺, C₅₈H₁₁₀O₆+OH, C₄₀H₇₄O₅+NH₄⁺, and combinations thereof.

10. The method according to claim 7, wherein the lipid markers are selected from the group consisting of: DG (18:0/18:0), TG (18:0/18:0/18:1), TG(12:0/14:0/16:0), TG (18:0/18:0/19:1)+OH, TG (18:0/18:1/19:0)+OH, DG(18:2/19:0), and combinations thereof.

11. The method according to claim 1, wherein the PRMD is selected from the group consisting of: hypocalcemia, fatty liver syndrome, ketosis, retained placenta, obturator nerve paresis (OP), and displaced abomasum.

12. The method according to claim 4, wherein cows with elevated levels of lipid markers having a mass to charge ratio of about 344.23, 570.46, 682.56, and/or 855.75, as compared to levels in normal controls, have an increased risk of PRMD's.

13. The method according to claim 4, wherein cows with decreased levels of a lipid marker having a mass to charge ratio of about 586.54, as compared to levels in normal controls, have an increased risk of PRMD's.

14. The method according to claim 8, wherein cows with decreased levels of a lipid marker having a mass to charge ratio of about 642.56 and/or 740.67, as compared to levels in normal controls, have an increased risk of PRMD's.

15. The method according to claim 8, wherein cows with increased levels of a lipid marker having a mass to charge ratio of about 906.84, as compared to levels in normal controls, have an increased risk of PRMD's.

16. A method of analyzing lipid profiles in cattle, the method comprising:
obtaining a colostrum or milk sample from a post-partum cow 0-7 days after parturition;
extracting lipids from the sample; and
analyzing the sample using mass-spectrometry for levels of one or more lipid markers.

17. A panel of lipid markers of production-related metabolic disease in milk or colostrum from 0-7 days post-parturient cattle, the panel comprising one or more lipid markers having a mass to charge ratio selected from the group consisting of: about 344.23, 570.46, 586.54, 642.56, 652.55, 682.56, 740.67, 855.75, 906.84, 919.83, and combinations thereof, as determined by electrospray injection time-of-flight mass-spectrometry (ESI-TOF MS).

18. The panel of lipid markers of claim 17, wherein the lipid markers have an elemental composition selected from the group consisting of: C₄₀H₅₆O+NH₄⁺, C₃₅H₆₈O₅+NH₄⁺, C₅₅H₉₈O₆+H⁺, C₃₉H₇₆O₅+NH₄⁺ and C₅₇H₁₀₈O₆+NH₄⁺, C₄₁H₇₆O₆+NH₄⁺, C₂₁H₂₆O₃+NH₄⁺, C₅₇H₁₀₈O₆+NH₄⁺, C₅₈H₁₁₀O₆+OH, C₄₀H₇₄O₅+NH₄⁺, C₄₅H₈₆O₆+NH₄⁺, and combinations thereof.

19. The panel of lipid markers of claim 17, wherein the lipid markers are present in milk 4-7 days after parturition.

20. The panel of lipid markers of claim 17, wherein the lipid markers are present in colostrum 0 to 24 hours after parturition.

21. The method of claim 16, wherein analyzing the sample comprises using electrospray injection time-of-flight mass-spectrometry (ESI-TOF MS).
